(19) United States
(12) Patent Application Publication
(10) Pub. No.: US 2022/0195385 A1
(43) Pub. Date: Jun. 23, 2022

(54) MOTILE CELL SORTING DEVICE

(71) Applicants: The University of Tokyo, Tokyo (JP); St. Marianna University School of Medicine, Kawasaki-shi, Kanagawa (JP); International University of Health and Welfare, Ohtawara-shi, Tochigi (JP); Japanese Organization for Medical Device Development, Inc., Tokyo (JP)

(72) Inventors: Kazuhiro KAWAMURA, Narita-shi, Chiba (JP); Masashi IKEUCHI, Tokyo (JP); Keiji KUBO, Tokyo (JP)

(73) Assignees: The University of Tokyo, Tokyo (JP); St. Marianna University School of Medicine, Kawasaki-shi, Kanagawa (JP); International University of Health and Welfare, Ohtawara-shi, Tochigi (JP); Japanese Organization for Medical Device Development, Inc., Tokyo (JP)

(21) Appl. No.: 17/598,845

(22) PCT Filed: Mar. 27, 2020
(86) PCT No.: PCT/JP2020/014023

§ 371 Date: Sep. 27, 2021

(30) Foreign Application Priority Data
Mar. 28, 2019 (JP) 2019-064636

(51) Int. Cl. C12N 5/071; C12M 1/26; C12M 1/34; C12Q 1/24; B01L 3/00

(52) U.S. Cl. CPC C12N 5/0612; C12M 1/26; C12M 1/34; B01L 2300/0864; B01L 3/502761; B01L 2200/0652; B01L 2300/0816; C12Q 1/24

(57) ABSTRACT

[Problem to be Solved] Provided is a motile cell sorting device that can effectively sort motile cells, such as sperms, having excellent motility and morphology by performing image analysis when animal cells are sorted. [Means for Solution] The motile cell sorting device 1 comprises a storage unit 3, a first introduction unit (cell injection channel) 5, a first introduction control unit 7, a first holding unit (cell capture area) 9, a first derivation unit (cell collection channel) 11, a first derivation control unit 13, a collection unit (liquid storage) 15, a photographing unit 17, and a first controller 19.

### TECHNICAL FIELD

This invention relates to a sorting device for motile cells, such as sperms, and a sorting method using the device.

### BACKGROUND ART

JP2016-514955A discloses a system for sorting sperms. This system is intended to collect functional sperms using a fine pore that is close to the passage of the female external genitalia.

JP6196614B discloses analysis and sorting of motile cells.

### CITATION LIST

### Patent Literature

PTL 1: JP2016-514955A
PTL 2: JP6196614B

### SUMMARY OF THE INVENTION

### Technical Problem

The systems disclosed in JP2016-514955A and JP6196614B have a problem that functional sperms cannot be effectively sorted. Further, these systems also have a problem that even deformed sperms etc. are sorted.

Accordingly, an object of the present invention is to provide a device and method that can effectively sort motile cells, such as sperms, having excellent motility and morphology.

### Solution to Problem

The present invention is basically based on the finding that motile cells, such as sperms, having excellent motility and morphology can be effectively sorted by performing image analysis when motile cells are sorted.

One of the embodiments described in this specification relates to a motile cell sorting device.

The motile cell sorting device 1 comprises a storage unit 3, a first introduction unit 5, a first holding unit (cell capture area) 9, a first derivation unit 11, a first derivation control unit 13, a collection unit (liquid storage) 15, a photographing unit 17, and a first controller 19.

The storage unit 3 is an element for storing a plurality of motile cells.

The first introduction unit 5 refers to a portion in which the motile cells stored in the storage unit 3 are introduced into the first holding unit.

The first holding unit 9 is a storage portion (e.g., a cell capture area) in which the motile cells having passed through the first introduction unit are held movably. The holding unit 9 does not have to be a single container, and may be a part continuous with the introduction unit.

The first derivation unit 11 is a derivation unit (e.g., a cell collection channel) to which the motile cells held in the first holding unit are derived.

The first derivation control unit 13 is an element (e.g., a valve) for controlling the open and closed state of the first holding unit 9 and the first derivation unit 11.

The collection unit 15 is an element (e.g., a liquid storage) connected to the first derivation unit, and for collecting and storing the sorted motile cells.

The photographing unit 17 is an element for photographing the animal cells held in the first holding unit.

The first controller 19 is an element for analyzing the animal cells photographed by the photographing unit, and controlling the opening and closing of the first derivation control unit according to the analysis results.

The motile cell sorting device 1 may further comprise a first introduction control unit 7 that controls the open and closed state of the storage unit and the first introduction unit.

Moreover, the motile cell sorting device 1 may further comprise a first introduction path (cell injection channel) 8 connecting the storage unit 3 and the first introduction unit such that the motile cells are movable.

Furthermore, the motile cell sorting device 1 may further comprise a first derivation path connecting the first derivation unit 11 and the collection unit 15 such that the motile cells are movable.

The motile cell sorting device 1 preferably comprises a plurality of introduction units, a plurality of introduction control units, a plurality of holding units, a plurality of derivation units, a plurality of derivation control units, and a plurality of controllers. The plurality of introduction control units, the plurality of holding units, the plurality of derivation units, the plurality of derivation control units, and the plurality of controllers correspond to the respective introduction units, and can perform the same operation as that of the motile cell sorting device 1 described above.

In a preferable example of the above device,

the controller comprises a normal cell morphology memory unit that stores data relating to the morphology of normal animal cells;

the controller determines an evaluation value relating to the morphology of the animal cells photographed by the photographing unit, using data relating to the morphology of the animal cells photographed by the photographing unit, and the data relating to the morphology of the normal animal cells stored in the normal cell morphology memory unit; and

the analysis results include the evaluation value relating to the morphology.

In a preferable example of the above device,

the controller further comprises a normal cell motility memory unit that stores data relating to the motility of normal animal cells;

the controller determines an evaluation value relating to the motility of the animal cells photographed by the photographing unit, using data relating to the motion of the animal cells photographed by the photographing unit, and data relating to the motion of the normal animal cells stored in the normal cell motility memory unit; and

the analysis results further include the evaluation value relating to the motility.

In a preferable example of the above device,

the controller further comprises:
a normal cell specific index memory unit that stores data relating to a specific index other than the morphology of normal animal cells and the motility, and
a specific index update unit for updating the data relating to the specific index by machine learning;

the controller determines an evaluation value relating to a specific index of the animal cells photographed by the photographing unit, using data relating to the specific index of the animal cells photographed by the photographing unit, and the data relating to the specific index of the normal animal cells stored in the normal cell specific index memory unit; and

the analysis results further include the evaluation value relating to the specific index.

In a preferable example of the above device, the motile cells are sperms. Further, it is preferable that one sperm is introduced into the first introduction unit at a time.

One of the embodiments described in this specification relates to a method for sorting motile cells with excellent motility and morphology.

This method comprises the following steps.

First motile cells stored in a storage unit that stores a plurality of motile cells are introduced through a first introduction unit into a first holding unit that can hold the first motile cells movably.

The first animal cells held in the first holding unit are photographed.

The first animal cells photographed in the photographing step are analyzed by a controller, and a first derivation control unit is opened according to the analysis results.

After the first derivation control unit is brought into an open state, the first motile cells held in the first holding unit are derived to the first derivation unit.

The first motile cells having passed through the first derivation unit are collected into a collection unit.

Only when the controller determines that the first animal cells are excellent in motility and morphology, the derivation control unit is opened and the first motile cells are collected; thus, motile cells with excellent motility and morphology can be effectively collected.

### Advantageous Effects of the Invention

It is possible to provide a device and method that can effectively sort motile cells, such as sperms, having excellent motility and morphology.

### BRIEF DESCRIPTION OF THE DRAWINGS

FIG. 1 is a conceptual diagram for explaining a motile cell sorting device.

FIG. 2 is a conceptual diagram that combines layers constituting the motile cell sorting device.

FIG. 3 is a diagram for explaining a configuration example of a cell injection channel layer having an introduction path, a valve layer having a storage unit and a valve, and a cell collection channel layer having a derivation path.

FIG. 4 is a conceptual diagram showing how sperms are selectively guided to a derivation path.

FIG. 5 is a conceptual diagram showing the entire configuration of a system in Examples.

FIG. 6 shows a photograph replacing a drawing of a device having a 1×2 model substrate.

FIG. 7 shows a photograph replacing a drawing of a device having a 10×10 model substrate.

FIG. 8 shows photographs replacing drawings showing a production example of a valve layer.

FIG. 9 shows a portion that determines sperms in Example 2. The left side of FIG. 9 is a sorting part, and the right side of FIG. 9 is a conceptual diagram showing, by an image, the presence of sperms in holding units.

FIG. 10 is a conceptual diagram showing the layer configuration of the system of Example 2.

### DETAILED DESCRIPTION OF THE INVENTION

Modes for carrying out the present invention will be described below using the drawings. The present invention is not limited to the modes described below, and includes those appropriately modified from the following modes within the scope obvious to those skilled in the art.

One of the embodiments described in this specification relates to a motile cell sorting device.

Examples of motile cells include sperm cells of mammals (e.g., humans or non-human mammals). An example of the motile cell sorting device is a device for sorting, from the collected sperms, sperms with excellent motility and morphology for use in fertilization. The following mainly describes sperms; however, the motile cells are not limited to sperms.

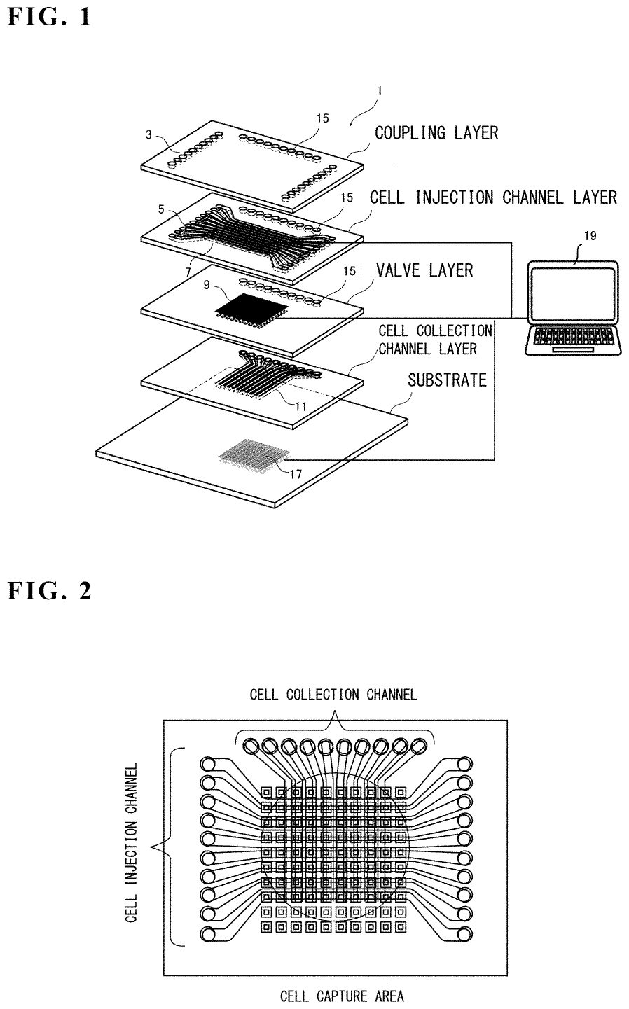

FIG. 1 is a conceptual diagram for explaining a motile cell sorting device. In the example of FIG. 1, the motile cell sorting device comprises a plurality of layers that are combined to constitute one device. The motile cell sorting device is not limited to one comprising a plurality of layers. However, a motile cell sorting device comprising a plurality of layers is preferable because channels can be controlled in consideration of the fluidity of sperms. FIG. 1 shows each layer in a separate state.

FIG. 2 is a conceptual diagram that combines layers constituting the motile cell sorting device. As shown in FIG. 2, when the motile cell sorting device is viewed from the upper surface, it is preferable that there are a plurality of storage units 3 on the periphery of the upper surface, and that there are introduction units 5 that connect the storage units 3 and holding units 9 toward a cell capture area (area where the holding units are present on the matrix) present in the central area. It was basically difficult to identify sperms with conventional devices. On the other hand, the device of this embodiment with such a configuration can efficiently guide many sperms to the holding units 9, and can prevent the failure to capture the target sperms. According to this embodiment, the amount of solution that can be treated at one time and the throughput can be increased by using multiple channels and a multivalve structure. The “multiple channels” mean that there are a plurality of introduction units, and the “multivalve structure” means that there are a plurality of holding units. In particular, many sperms can be sorted at the same time by integrating holding units into a matrix.

The motile cell sorting device 1 comprises a storage unit 3, a first introduction unit 5, a first introduction control unit 7, a first holding unit (cell capture area) 9, a first derivation unit (cell collection channel) 11, a first derivation control unit 13, a collection unit (liquid storage) 15, a photographing unit 17, and a first controller 19.

The storage unit 3 is an element for storing a plurality of motile cells. The storage unit may have a volume, for example, that can store the collected semen. The motile cell sorting device may have only one storage unit or a plurality of storage units. In the example of FIG. 1, the storage units are present in a coupling layer (first layer). The storage unit may simply be a portion that introduces sperms.

The first introduction unit 5 refers to a portion into which the motile cells stored in the storage unit 3 are introduced. This portion may be a channel (e.g., a cell injection channel) or a hole. Further, this portion may be a part of the holding unit into which the motile cells are introduced (e.g., inlet portion). Some of the motile cells stored in the storage unit 3 move to the first introduction unit when the first introduction unit is brought into an open state. The introduction unit may be, for example, a channel provided on a substrate, or may be holes on a plurality of substrates. The channels, such as introduction units, are processed such that, for example, sperms can move. Such channels themselves in which sperms can move are already known, as described in JP6196614B, for example. For example, when the motile cells are sperms, in order for one sperm to move without being damaged, the introduction unit may be a hole, and the diameter of the hole may be 1 μm or more and 20 μm or less, or 5 μm or more and 10 μm or less. It is preferable that the introduction unit is filled with a solution such that the sperms can move. The first introduction unit 5 may be connected to the storage unit 3, for example, through a valve. The valve allows the introduction of the sperms one by one into the introduction unit. However, even without the presence of a valve, if the diameter of the hole does not allow a plurality of sperms to be guided, the sperms are introduced into the storage unit 3 one by one; thus, no valve is necessary particularly in that case. Then, when the storage unit 3 and the first introduction unit 5 are brought into an open state, the sperms stored in the storage unit 3 are introduced into the first introduction unit 5. The motile cell sorting device preferably has a plurality of (2 or more, for example, 2 or more and 100 or less, 8 or more and 50 or less, or 12 or more and 45 or less) introduction units. Storage units may be present corresponding to the respective introduction units, or a plurality of introduction units may be connected to one storage unit. In the example of FIG. 1, the introduction units are present in a cell injection channel layer. When viewed from the upper surface of the device, the introduction units are configured to guide the sperms stored in the storage units to the cell capture area (area where the holding units are present) positioned in the center of the device. This makes it possible to photograph the cell capture area from the upper or lower side of the device.

Even when there are plurality of storage units, the first introduction unit 5 is not necessarily a plurality of channels. For example, a spacer may be provided between the coupling layer and the valve layer such that the gap between the coupling layer and the valve layer is 10 μm or more and 40 μm or less, or 15 μm or more and 30 μm or less, and the gap between the coupling layer and the valve layer may be filled with a solution in which the sperms can move. In introduction methods using conventional micro-channels, irregularities and connected parts present in channels caused damage to sperms. When thus using channels that are like a large field of sea, free-swimming sperms can move to the storage unit 9. In this case, an introduction control unit, described below, may not be present. There may be a plurality of introduction units.

The first introduction control unit 7 is an optional element for controlling the open and closed state of the storage unit and the first introduction unit. The first introduction control unit 7 preferably performs control such that one sperm is introduced into the first introduction unit. In this respect, for example, the presence or absence of sperms may be determined by photographing the state of the introduction unit, and performing image analysis. Then, when it is determined that there is no sperm in the introduction unit, the storage unit and the first introduction unit may be brought into an open state. In this manner, a single sperm can be derived to each introduction unit. For example, the connected part between the storage unit 3 and the introduction unit 5 may be connected through a microvalve whose diameter changes between 0.5 μm or more and 2 μm. This hole preferably has a diameter that increases or decreases by applying a voltage. A suction device, such as a vacuum pump, is connected to the introduction unit side, and when the first introduction control unit 7 controls the introduction unit side to a negative pressure, the sperms present in the storage unit can be introduced into the introduction unit one by one. Such control can be achieved by MEMS plate control.

The first introduction control unit 7 may be a suction system that suctions the motile cells, such as sperms, stored in the storage unit 3. The suction system may be any system as long as it can control the holding unit 9 to a negative pressure. When the holding unit 9 is brought into a negative pressure by the suction system, the motile cells, such as sperms, stored in the storage unit 3 are sucked and stored in the holding unit 9 via the introduction unit. Suction is stopped after the sperms etc. are stored in the holding unit 9, whereby it is possible to perform control such that the next sperms are not sucked into the holding units 9. In this case, for example, the introduction unit is preferably a microvalve.

The first holding unit 9 is a storage portion in which the motile cells having passed through the first introduction unit are held movably. The sperms derived to the introduction unit are captured in this storage portion. When the motile cell sorting device has a plurality of introduction units, there are preferably holding units corresponding to the respective introduction units. The plurality of holding units are preferably provided in a layer (e.g., cell capture area) of the motile cell sorting device. When such a layer is provided, each holding unit can be easily photographed by using a photographing unit, described later. The holding unit 9 does not have to be a single container, and may be a part continuous with the introduction unit. The number of holding units does not have to be one, and a plurality of holding units may be present in one device. It is preferable that a plurality of holding units are present in one device. A plurality of holding units may be present, for example, on grid points, and may be present on grid points of a square grid, a rectangular grid, a centered rectangular grid, an orthorhombic grid, or a hexagonal grid, or may be present at random. It is preferable that the center of a plurality of holding units is present on a grid point of a rectangular grid, a centered rectangular grid, an orthorhombic grid, or a hexagonal grid, because the photographed image can be easily discriminated.

The first derivation unit 11 is a derivation unit (e.g., a cell collection channel) to which the motile cells held in the first holding unit are derived. The derivation unit may be one to which the sperms can be derived. In the examples of FIGS. 1 and 2, the derivation unit is connected to the first surface through a plurality of layers. The derivation unit 11 may be an outlet portion of the holding unit 9, or may be a hole or a channel. This is similar to the previously explained introduction unit, and a similar configuration can be employed. There may also be a plurality of derivation units.

The first derivation control unit 13 is an element for controlling the open and closed state of the first holding unit 9 and the first derivation unit 11. The first holding unit 9 and the first derivation unit 11 are connected, for example, by an open/close control element, such as a valve. The open/close control element can be controlled to bring the first holding unit 9 and the first derivation unit 11 into an open state or a closed state. For example, when a controller, described later, determines that the sperms in the holding unit are excellent in morphology and motility, the open/close control element is brought into an open state. Then, the sperms held in the holding unit are derived to the derivation unit. When the sperms held in the holding unit are derived to the derivation unit, the corresponding introduction unit and storage unit may be brought into an open state such that new sperms are contained in the holding unit. For example, machine learning and time correlation image analysis may be performed using software to analyze the morphology and motion of the sperms in the holding unit 9. Conventional devices had a problem, for example, that even through sperms with high motility could be derived, many deformed sperms were included. According to the device of this embodiment, it is possible to eliminate deformed sperms with high accuracy and isolate only the target sperms with excellent morphology by efficiently photographing sperms and utilizing judgment means by machine learning. In addition, software processing through machine learning etc. makes it possible to sort excellent sperms in a more efficient and accurate manner than when a person sorts sperms while viewing images. Sperms with excellent motility can be sorted by performing time correlation image analysis. In other words, a plurality of images of sperm present in a holding unit photographed by the photographing unit are collected over time, and the motion of the sperm is analyzed, whereby the motility of the sperm present in the holding unit can be determined.

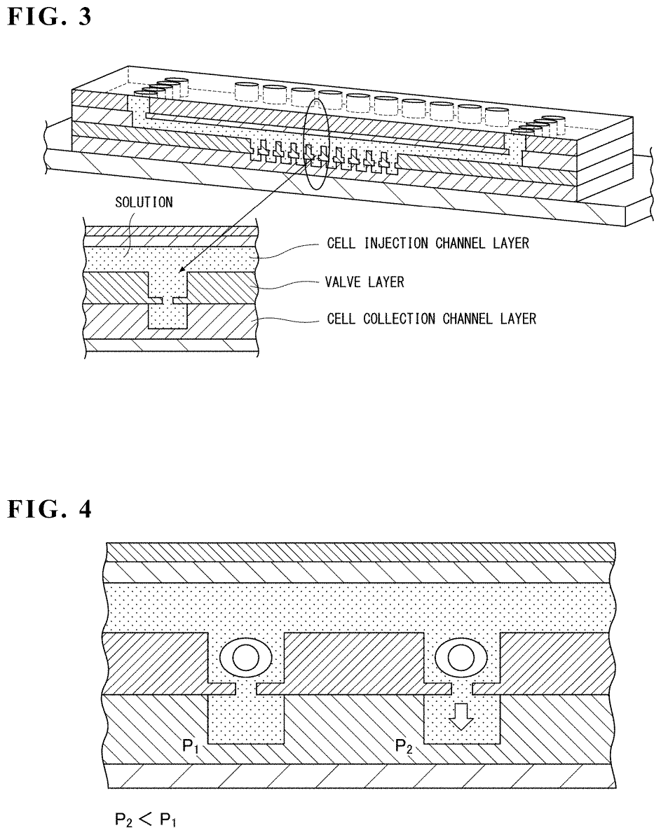

FIG. 3 is a diagram for explaining a configuration example of a cell injection channel layer having an introduction unit, a valve layer having a storage unit and a valve, and a cell collection channel layer having a derivation unit. For example, when the derivation control unit 13 guides the sperms present in the holding unit 9 to the derivation unit 11, the holding unit 9 and the derivation unit 11 may be brought into an open state by setting the diameter of the connected part (valve part) between them to a size that allows the sperms to pass through.

FIG. 4 is a conceptual diagram showing how sperms are selectively guided to the derivation unit. In this example, similarly as described above, the sperms can be guided to the derivation unit 11 side by bringing the derivation unit 11 side to a negative pressure.

The collection unit 15 is an element (e.g., a liquid storage) connected to the first derivation unit, and for collecting and storing the sorted motile cells. The collection unit 15 is preferably configured to be able to derive the sorted sperms to the outside. In the examples of FIGS. 1 and 2, the collection unit is present in the first layer of the device, and is connected to the derivation unit through a plurality of layers. The sperms present in the collection unit are collected, whereby sperms with excellent motility and morphology can be collected.

The photographing unit 17 is an element for photographing the animal cells held in the first holding unit. The photographing unit 17 is configured to be able to output the photographed image data to a controller, described later. The photographing unit 17 is, for example, a CCD camera, and can photograph images while associating the address of each holding unit with picture elements, such as pixels, and output the data to the controller. When the cell collection channel layer (and substrate) is transparent or translucent, the sperms held in the holding unit present in the valve layer can be photographed from the cell collection channel layer (and substrate) side.

The first controller 19 is an element for analyzing the animal cells photographed by the photographing unit, and controlling the opening and closing of the first derivation control unit according to the analysis results. The controller is, for example, a computer. The computer has an input/output unit, a control unit, a calculation unit, and a memory unit. The elements are each connected by a bus or the like such that information can be exchanged. The memory unit stores programs, as appropriate. Information is input from the input/output unit. Then, the control unit reads various data from the memory unit based on the commands of the programs stored in the memory unit. Then, the calculation unit is allowed to perform various calculations using the input data and the data read from the memory unit. The results determined by the calculation unit are appropriately stored in the memory unit and output from the input/output unit. The controller is connected to the photographing unit, the introduction control unit, and the derivation control unit such that information can be exchanged. The image data output from the photographing unit are stored, as appropriate, in the memory unit within the controller, and subjected to various types of calculation processing, such as image analysis processing. Further, the control commands from the controller are output to the introduction control unit and the derivation control unit, and the introduction control unit and the derivation control unit perform various controls according to the control commands from the controller.

The motile cell sorting device 1 preferably comprises a plurality of introduction units, a plurality of introduction control units, a plurality of holding units, a plurality of derivation units, a plurality of derivation control units, and a plurality of controllers. The plurality of introduction control units, the plurality of holding units, the plurality of derivation units, the plurality of derivation control units, and the plurality of controllers correspond to the respective introduction units, and can perform the same operation as that of the motile cell sorting device 1 described above.

In a preferable example of the above device, the controller comprises a normal cell morphology memory unit that stores data relating to the morphology of normal animal cells.

The controller determines an evaluation value relating to the morphology of the animal cells photographed by the photographing unit, using data relating to the morphology of the animal cells photographed by the photographing unit, and the data relating to the morphology of the normal animal cells stored in the normal cell morphology memory unit. The analysis results include the evaluation value relating to the morphology. The controller issues a command, for example, to the derivation control unit. The derivation control unit 13 controls the open and closed state of the first holding unit 9 and the first derivation unit 11. For example, the controller outputs a control command to the derivation control unit 13 so as to bring the first holding unit 9 and the first derivation unit 11 into an open state. According to the control command, the derivation control unit 13 brings the first holding unit 9 and the first derivation unit 11 into an open state. Since the fact that sperms can move each channel means that the sperms have motility, sperms with morphology and motility can be sorted by evaluating their morphology.

In a preferable example of the above device, the controller further comprises a normal cell motility memory unit that stores data relating to the motility of normal animal cells. The controller determines an evaluation value relating to the motility of the animal cells photographed by the photographing unit, using data relating to the motion of the animal cells photographed by the photographing unit, and data relating to the motion of the normal animal cells stored in the normal cell motility memory unit. The analysis results further include the evaluation value relating to the motility. For example, the controller analyzes the momentum of sperms from sperm images photographed by the photographing unit, and obtains an evaluation value relating to the momentum (motility). The memory unit stores a threshold value relating to the evaluation value of motility. The control unit causes the calculation unit to perform a calculation to compare the determined evaluation value of motility with the threshold value of the evaluation value of motility stored in the memory unit. Then, when the evaluation value of motility exceeds the threshold value relating to the evaluation value of motility, it is determined that the motility is acceptable. Then, the memory unit stores an evaluation value relating to the morphology. As a result of the same processing as for the motility, when the evaluation value relating to the morphology determined above is larger than a threshold value of the evaluation value relating to the morphology, it is determined that the morphology is also acceptable. The determination results are stored in the memory unit, as appropriate. The controller may read the results of acceptance or rejection stored in the memory unit, and may output a control command to the derivation control unit 13 so as to bring the first holding unit 9 and the first derivation unit 11 into an open state when the motility and the morphology are acceptable. In this manner, sperms with excellent motility and morphology are sorted.

In a preferable example of the above device, the controller further comprises a normal cell specific index memory unit that stores data relating to a specific index other than the morphology of normal animal cells and the motility, and a specific index update unit for updating the data relating to the specific index by machine learning. The specific index is color, for example. The success rate of fertilization may be analyzed by machine learning using data relating to the color of sperm and the success rate of fertilization, and the combination of morphology, motility, and color, so that the one with a high fertilization rate can be selected. In this case, the controller determines an evaluation value relating to a specific index of the animal cells photographed by the photographing unit, using data relating to the specific index of the animal cells photographed by the photographing unit, and the data relating to the specific index of the normal animal cells stored in the normal cell specific index memory unit. The analysis results further include the evaluation value relating to the specific index.

When it is determined that the motile cells are not preferable (the threshold values are not exceeded), the holding unit may be brought into a closed state to prevent new motile cells from entering, or the motile cells may be introduced into a collection path that collects unsuitable motile cells, or may be returned to the storage unit.

One of the embodiments described in this specification relates to a method for sorting motile cells with excellent motility and morphology.

This method comprises the following steps.

First motile cells stored in a storage unit that stores a plurality of motile cells are introduced through a first introduction unit into a first holding unit that can hold the first motile cells movably.

The first motile cells held in the first holding unit are photographed by a photographing unit.

The first motile cells photographed by the photographing unit are analyzed by a controller, and a first derivation control unit is opened according to the analysis results.

After the first derivation control unit is brought into an open state, the first motile cells held in the first holding unit are derived to a first derivation unit.

The first motile cells having passed through the first derivation unit are collected into a collection unit.

In this manner, the motile cells analyzed based on photographing are collected. In particular, only when the controller determines that the first animal cells are excellent in motility and morphology, the derivation control unit is opened and the first motile cells are collected; thus, motile cells (e.g., sperms) with excellent motility and morphology can be effectively collected.

### Example 1

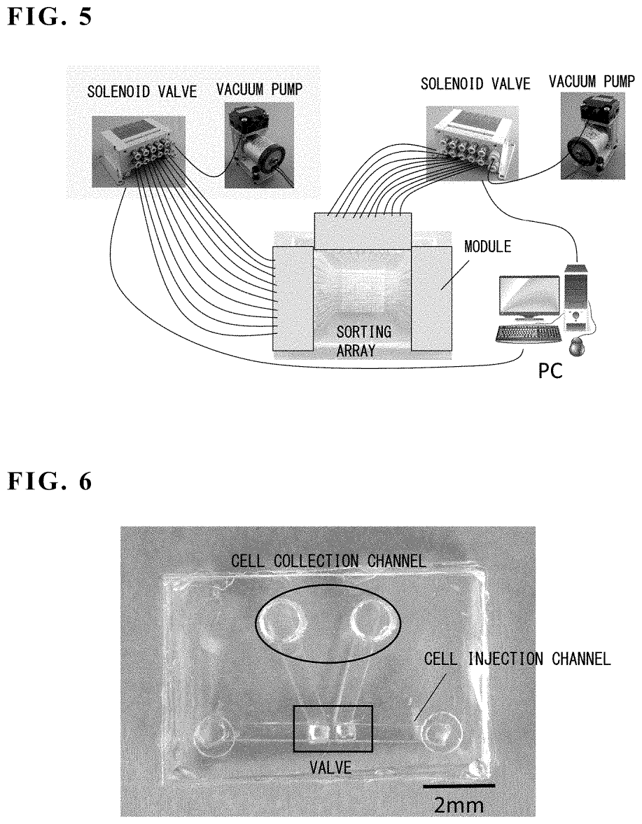

FIG. 5 is a conceptual diagram showing the entire configuration of a system in Examples. In this example, vacuum pumps are used to introduce and derive sperms, image photographing is performed by a CCD camera (not shown), and various controls are performed by a PC (computer).

FIG. 6 shows a photograph replacing a drawing of a device having a 1×2 model substrate. Using this model device, it was confirmed that sperms were sorted properly, and shooting was performed as a video.

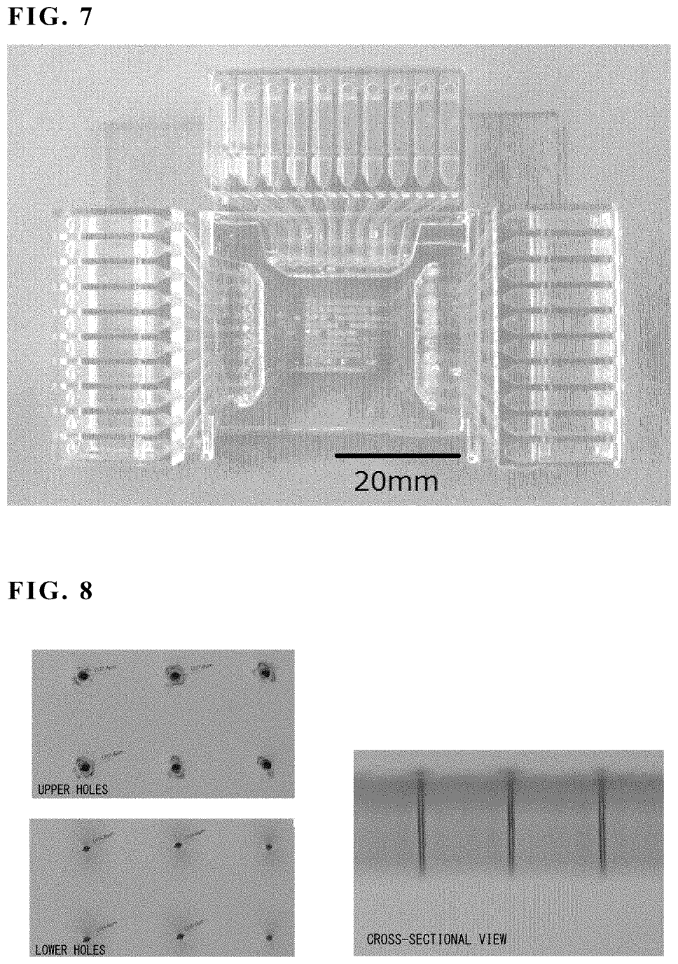

FIG. 7 shows a photograph replacing a drawing of a device having a 10×10 model substrate. Using this model device, it was confirmed that sperms were sorted properly, and shooting was performed as a video.

FIG. 8 shows photographs replacing drawings showing a production example of a valve layer. The valve layer was produced using non-alkali glass, and the thickness was 100 μm. The upper-left photograph shows upper holes, and the lower-left photograph shows lower holes. The diameter of the upper holes was about 7 to 8 μm. On the other hand, the diameter of the lower holes was about 4 to 5 μm. The right photograph is a cross-sectional view. 2500 (50×50) of such holes were provided. The distance between the holes (distance between the grids) was 100 μm (this distance can be adjusted, for example, from 10 μm or more to 500 μm or less, or from 50 μm or more to 200 μm or less).

### Example 2

Next, an example of a sperm sorting device using a microvalve is shown.

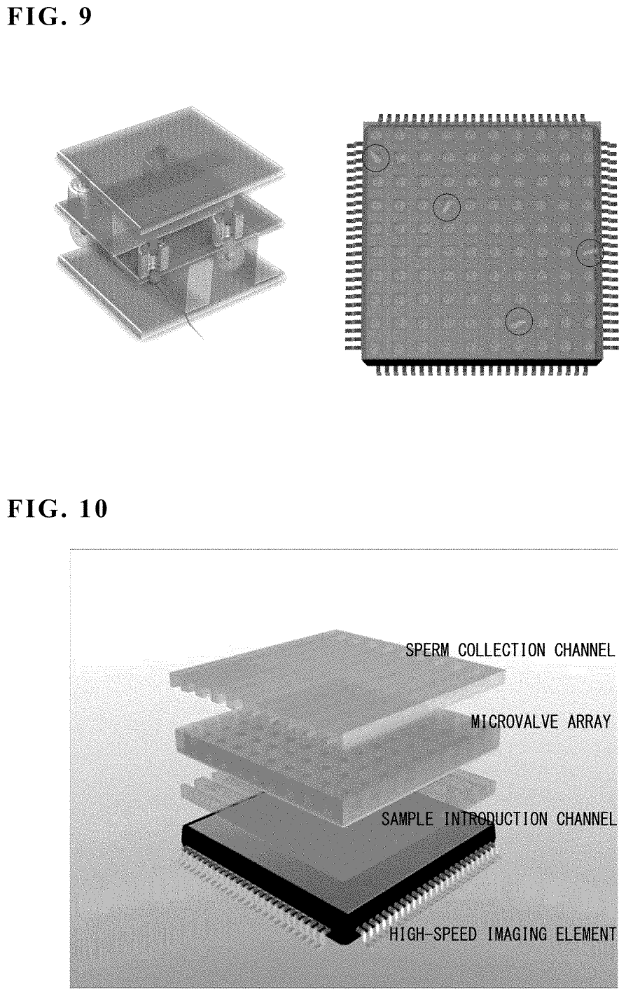

FIG. 9 shows a portion that determines sperms in Example 2. The left side of FIG. 9 is a sorting part, and the right side of FIG. 9 is a conceptual diagram showing, by an image, the presence of sperms in holding units. Cells (holding units) for which it is determined, as a result of image analysis, that sperms are present are indicated by circles. Then, image analysis is performed to determine whether the sperms present in the cells exceed a threshold value. FIG. 10 is a conceptual diagram showing the layer configuration of the system of Example 2. In the example of FIG. 10, a high-speed imaging element layer, a sample introduction channel (storage unit), a microvalve array (a plurality of holding units), and a sperm collection channel are present from lower layers.

### INDUSTRIAL APPLICABILITY

The present invention can be used, for example, as a sperm sorter, and thus can be utilized in the medical device industry.

The present invention can sort desired motile cells to be used, and thus can be utilized in the laboratory equipment industry.

### REFERENCE SIGNS LIST

1. Motile cell sorting device
3. Storage unit
5. Introduction unit
7. Introduction control unit
9. Holding unit
11. Derivation unit
13. Derivation control unit
15. Collection unit
17. Photographing unit
19. Controller

What is claimed is:

1. A motile cell sorting device, comprising:
a storage unit that stores a plurality of motile cells;
a first introduction unit connected to the storage unit, and into which the motile cells stored in the storage unit are introduced;
a first holding unit that movably holds the motile cells having passed through the first introduction unit;
a first derivation unit to which the motile cells held in the first holding unit are derived;
a first derivation control unit that controls an open and closed state of the first holding unit and the first derivation unit;
a collection unit connected to the first derivation unit;
a photographing unit that photographs the motile cells held in the first holding unit; and
a first controller that analyzes the motile cells photographed by the photographing unit, and controls opening and closing of the first derivation control unit according to analysis results,
wherein the motile cells are sperms, and one sperm is introduced into the first introduction unit.

2. The device according to claim 1, further comprising
a first introduction control unit that controls an open and closed state of the storage unit and the first introduction unit.

3. The device according to claim 1, further comprising:
a first introduction path connecting the storage unit and the first introduction unit such that the motile cells are movable; and
a first derivation path connecting the first derivation unit and the collection unit such that the motile cells are movable.

4. The device according to claim 1, wherein
the first holding unit is a storage portion in which the motile cells having passed through the first introduction unit are captured and held movably.

5. The device according to claim 4, wherein
the first controller analyzes the morphology and motility of the motile cells held in the first holding unit and photographed by the photographing unit.

6. The device according to claim 4, wherein
when the first controller determines that the motile cells held in the first holding unit and photographed by the photographing unit are excellent in morphology and motility, the first derivation control unit is brought into an open state.

7. The device according to claim 1, wherein
the first controller comprises a normal cell morphology memory unit that stores data relating to the morphology of normal motile cells,
the first controller determines an evaluation value relating to the morphology of the motile cells photographed by the photographing unit, using data relating to the morphology of the motile cells photographed by the photographing unit, and the data relating to the morphology of the normal motile cells stored in the normal cell morphology memory unit, and
the analysis results include the evaluation value relating to the morphology.

8. The device according to claim 5, wherein
the first controller further comprises a normal cell motility memory unit that stores data relating to the motility of normal motile cells,
the first controller determines an evaluation value relating to the motility of the motile cells photographed by the photographing unit, using data relating to the motion of the motile cells photographed by the photographing unit, and data relating to the motion of the normal motile cells stored in the normal cell motility memory unit, and
the analysis results further include the evaluation value relating to the motility.

9. The device according to claim 6, wherein
the first controller further comprises:
a normal cell specific index memory unit that stores data relating to a specific index, the data relating to the specific index being data relating to an index other than the morphology of normal motile cells and other than the motion of the motile cells; and
a specific index update unit that updates the data relating to the specific index by machine learning,
the first controller determines an evaluation value relating to a specific index of the motile cells photographed by the photographing unit, using data relating to the specific index of the motile cells photographed by the photographing unit, and the data relating to the specific index of the normal motile cells stored in the normal cell specific index memory unit, and
the analysis results further include the evaluation value relating to the specific index.

10. A motile cell sorting method, comprising the steps of:
introducing first motile cells stored in a storage unit that stores a plurality of motile cells through a first introduction unit connected to the storage unit into a first holding unit that can hold the first motile cells movably;
photographing the first motile cells held in the first holding unit;
analyzing, by a controller, the first motile cells photographed in the photographing step, and opening a first derivation control unit according to analysis results;
after the first derivation control unit is brought into an open state, deriving the first motile cells held in the first holding unit to a first derivation unit; and
collecting the first motile cells having passed through the first derivation unit into a collection unit,
wherein the first motile cells are sperms, and one sperm is introduced into the first introduction unit.
